(19) United States
(12) Patent Application Publication
(10) Pub. No.: US 2014/0080739 A1
(43) Pub. Date: Mar. 20, 2014

(54) Nanobeads With Multiple Oriented Adapting Peptides For Binding To Capture Molecules

(71) Applicant: National Cheng Kung University, Tainan (TW)

(72) Inventor: Lynn Ling-Huei Huang, Tainan (TW)

(73) Assignee: National Cheng Kung University, Tainan (TW)

(21) Appl. No.: 13/622,396

(22) Filed: Sep. 19, 2012

(51) Int. Cl. G01N 33/543

(52) U.S. Cl. CPC G01N 33/54346
USPC 506/18

(57) ABSTRACT

Provided is a capture nanobead with multiple oriented adapting peptides, comprising: a nanobead; and multiple adapting peptides, each adapting peptide specifically recognizing an IgG constant region or fragment thereof and being chemically conjugated with the nanobead, whereby the adapting peptides are orientedly arranged on the nanobead. The capture nanobeads is capable of binding or conjugating with capture molecules such capture antibodies, which is useful as the basis to bind to alternative antibodies or Fab fragments in sandwich immunoassays.

### BACKGROUND OF THE INVENTION

1. Field of the Invention

The present invention relates to coated nanobeads with bonded or conjugated capture molecules, which is useful for detecting countering molecules or for sandwich immunoassay as the basis to bind to alternative antibodies, Fab fragments and so on.

2. Description of the Prior Arts

Sandwich immunoassay is useful for protein detection in the principle of antigen and antibody interaction. It is usually applied on plates in enzyme-linked immunosorbent assay (ELISA) as well as on surface of beads in a microchip using microscopy (Anal. Chem., 2000; 72: 1144) and flow cytometry. More advanced techniques such as multiplex bead-based immunoassays can detect a large number of proteins simultaneously in one well and have been applied for analysis of cytokines, growth factors, diseases biomarkers, intercellular molecules, etc. (J Immunol Methods., 1999; 277: 41; J Immunol Methods., 2000; 243: 243). Compared to plain wells in ELISA plates, beads enable more efficient, homogenous interaction between target proteins and capture antibody-coated beads in aqueous conditions.

The bead-based immunoassay is used as classical solid-phase sandwich immunoassays such as ELISA (Methods, 2006; 38: 294) and also includes capture antibody and reporter antibody, usually biotin-labeled which binds to streptavidin-labeled phycoerythrin (PE). Capture antibodies are covalently coupled on beads, instead of in wells of ELISA plate.

Micrometer sized fluorescent dyes-encoded polystyrene beads coated with various kinds of capture antibody against target protein can properly quantify proteins of interest in one sample tube. A conventional fluorometer/luminometer is performed for identifying various sets of the fluorescent/luminescent beads and measuring fluorescent/luminescent intensity of reporter antibody against antigen captured on the beads. Nowadays, instead of microbeads and for more efficient immunoassay in as little as 100 μL working solution, nanobeads are rarely investigated and their capacity for bead-based immunoassay is still not well known.

Nanobeads have attracted great interest in nanomedicine (Nano Today, 2008; 3: 40) for drug delivery system applications. Meanwhile, attributable to discovering fluorescent emission properties of metal nanocrystals, the nanocrystals are successfully used in protein labeling and protein tracking in cells (Science, 1998; 281: 2016). For protein quantification, gold nanocrystals, as small as 10-nm, coated with protein A was reported for an aggregation-based immunoassay using UV-Vis absorption spectroscopy (Anal. Chem., 2002; 74: 1624). A pioneer study of nanobead-based immunoassay was previously done using bacteria-producing metallic particles which were fused with staphylococcal protein A and coupled with capture antibody for detecting insulin in a 96-well microtiter plate using a luminometer (Anal. Chem., 2000; 72: 3518).

Besides of the nanobead application, detection instrument equipped with a fluidic system can meet the need of single bead detection. Nowadays, a flow cytometer is routinely used for cell characterization and biomarker detection. A few studies have already shown that the flow cytometer is potent for analyzing viruses (Cytometry, Part A, 2004; 57A: 94) and fluorescent beads (J. Control Release, 2009; 141: 328) as small as 100 nm. However, the application of nanobeads for bead-based immunoassay or affinity assay remains undeveloped. Insufficiency of technical means for demand on use of nanobeads in immunoassay, affinity assay, especially for fluidic detection system such as flow cytometry analysis, needs to be satisfied. To overcome the shortcomings of conventional immunoassay as well as affinity assay and provide a more efficient means, the present invention provides a nanobead conjugated with multiple adapting peptides, preferably in combination with capture molecules, to mitigate or obviate the aforementioned problems.

### SUMMARY OF THE INVENTION

The main objective to the present invention is to provide a nanobead having increased effectively the capacity of the capture molecular immobilization and also capable of being used to detect amount of countering molecules. In particular, the capture molecule is an antibody and the countering molecule is the antigen with specific affinity to the antibody. Such nanobeads can serve as sandwich immunoassay beads with minimum sample requirement. The present invention provides a capture nanobead with multiple oriented adapting peptides comprising: a nanobead; and multiple adapting peptides, each adapting peptide specifically recognizing a region on the capture molecule or fragment thereof and being chemically conjugated with the nanobead, whereby the adapting peptides are orientedly arranged on the nanobead.

Preferably, the adapting peptide specifically recognizes the constant region of an immunoglobulin (Ig).

Preferably, the region of the capture molecule is the constant region of the immunoglobulin; and more preferably, IgG.

According to the present invention, the term “adapting peptide” refers to any peptide, polypeptide, or protein specifically binding to the Ig constant region, IgG constant region, Ig gamma 1 chain C region, except for the integral protein A. The adapting peptide in accordance with the present invention includes, but is not limited to an Ig-binding domain of Protein A. As known in the art, Protein A is a surface protein originally found in the cell wall of the bacterium Staphylococcus aureus and composed of five homologous Ig-binding domains, each of which folds into a three-helix bundle and is able to bind immunoglobulins from many of mammalian species, reflecting on its high affinity to human IgG₁ and IgG₂ as well as mouse IgG₂ₐ and IgG_{2b} and moderate affinity to human IgM, IgA and IgE as well as to mouse IgG₃ and IgG₁. It does not react with human IgG₃ or IgD, nor will it react to mouse IgM, IgA or IgE.

Particularly, the adapting peptide as used hereby refers to molecules of monomer or dimer of anti-IgG affibody. According to the present invention, anti-IgG affibody consists of three alpha helices, one of Ig-binding domains of Protein A, and has 56 amino acids, naturally forming a dimer with a molecular weight of 14 kDa.

Preferably, the adapting peptides are anti-IgG affibodies.

According to the present invention, the adapting peptides are chemically conjugated with the nanobead, that is covalently coupled to the surface of the nanobead, in such as much as a chemical group of the anti-IgG affibody is connected to the surface of the nanobead via a bifunctional group of a cross-linking agent, whereby the adapting peptides are orientedly arranged on the nanobead.

According to the present invention, the term “orientedly arranged” refers to being arranged in a direction and in an order, such that the capture molecule in accordance with the present invention is capable of exposing an antigen recognition site to capture a target antigen.

Preferably, the nanobead in accordance with the present invention is adapted to binding for over 10 molecules of IgG per nanobead. Alternatively, the nanobead in accordance with the present invention is adapted to binding for over 50 μg/ml of IgG per 1.03×10⁸ μm².

The cross-linking agent in accordance with the present invention is selected from the group consisting of 1-ethyl-3-(3-dimethylaminopropyl)carboimide hydrochloride (EDC), an epoxide, a dialdehyde, a N-hydroxysuccinimide ester, a carbodiimide, genipin, a riboflavin, a flavonoid, a 6-maleimidohexanoic acid active ester, disuccinimidyl suberate, bis(sulfosuccinimidyl) suberate, and sulfosuccinimidyl-4-(N-maleimidomethyl)cyclohexane-1-carboxylate.

Preferably, the surface of the nanobead has carboxyl group; and the cross-linking agent is 1-ethyl-3-(3-dimethylaminopropyl)carboimide hydrochloride.

According to the present invention, the nanobead further comprises capture molecules. The capture molecules in accordance with the present invention respectively bind to the adapting peptides via specifically binding of the IgG constant region. The term “capture molecule” as used hereby refers to nature molecules or genetically engineered molecules containing a domain or a region such as an immunogolobulin gamma 1 constant region, which is recognized by the IgG-binding domain, binding to the adapting peptide in accordance with the present invention. For example, the capture molecule is mouse IgG. Alternatively, the capture molecule is human or mouse IgG₂ₐ labeled with fluorescein dye, wherein the fluorescein dye is selected from the group consisting of fluorescein isothiocyanate (FITC), phycoerythrin (PE), Texas Red and any other suitable dye for distinguishing the binding of the capture molecules as needed.

Particularly, the capture molecule in accordance with the present invention is any known capture antibody against a target antigen with or without conjugation to a dye, such as PE, FITC, Texas Red or any other known dye. More particularly, the capture molecule is PE-mouse IgG₂ₐ, anti-VEGFA mouse IgG₂ₐ.

Preferably, the capture molecules are covalently coupled with the adapting peptides by a cross-linking agent. The cross-linking agent as used hereby can be determined and used according to the properties of the adapting peptide and the capture molecules. Particularly, the suitable cross-linking agent herein is as previously described.

According to the present invention, the nanobead is a metallic particle, a polymeric particle, a ceramic particle or any other particles adapted to be conjugated with the adapting peptides in accordance with the present invention.

Preferably, the nanobead is a polystyrene bead, a natural resin bead, a synthetic resin bead, a silica bead or a gold bead.

More preferably, the nanobead is modified to contain a carboxyl group.

According to the present invention, the nanobead has a size between 10 nm to 900 nm. Preferably, the nanobead has a size between 20 nm to 500 nm. More preferably, the nanobead has a size between 50 nm to 200 nm.

The capture bead in accordance with the present invention is useful for nanobead-based sandwich immunoassay and can be used directly to detect a predetermined antigen or to capture an immunoglobulin-like molecules against a specific antigen. In the presence of the adapting molecules in accordance with the present invention, the capture bead is able to provide specific Ig-binding sites in a spatial orientation, instead of random arrangement through nonspecific conjugation. Thus, the capacity of binding to antigen per unit surface of the nanobead is promptly increased, reflecting on an improved fluorescent intensity or a lower detecting limit of the capture bead in accordance with the present invention.

Other objectives, advantages and novel features of the invention will become more apparent from the following detailed description when taken in conjunction with the accompanying drawings.

### BRIEF DESCRIPTION OF THE DRAWINGS

FIG. 1 is an illustrating scheme of anti-IgG affibody-conjugated 85-nm beads preparation, wherein the beads were activated with EDC and then conjugated with affibody molecules for further coating capture antibody and antigen detection in a nanobead-based immunoassay;

FIGS. 2A and 2B illustrates characterization of anti-IgG affibody-conjugated bead preparation:

FIG. 2A illustrates comparison of PE-labeled mouse IgG₂ₐ coupling intensities on 85-nm (solid circle) and 6-μm (solid triangle) beads, and PE-labeled mouse IgG₂ₐ capture intensities on anti-IgG affibody-conjugated 85-nm (open circle) and anti-IgG affibody-conjugated 6-μm (open triangle) beads (each detection using 2.6 μg beads, nearly with 4.55×10⁹ of 85-nm beads or 2.1×10⁴ of 6-μm beads respectively) detected using ELISA reader, wherein PE-labeled IgG₂ₐ concentrations were 0.5 ng/ml, 5 ng/ml, 50 ng/ml, 500 ng/ml, 5 μg/ml as well as 50 μg/ml;

FIG. 2B illustrates zeta potential of carboxyl polystyrene 85-nm beads and 6-μm beads (marked as*), wherein zeta potential of both beads were measured before and after EDC activation (EDC acti.), anti-IgG affibody conjugation (AF conj.), antibody (mouse IgG₂ₐ; anti-VEGFA monoclonal antibody) conjugation (Ab conj.) and antibody coated on anti-IgG affibody-conjugated sites (Ab-coated); for carboxyl beads and EDC-activated beads, n=3; others' beads, n=1, measured in duplicate; and the error bars represent means s.d;

FIGS. 3A and 3B show efficient binding PE-labeled mouse IgG₂ₐ on anti-IgG affibody-conjugated nanobeads and microbeads:

FIG. 3A illustrates comparison of PE-labeled IgG detection by anti-IgG affibody conjugated 85-nm beads (solid circle) and 6-μm beads (open triangle) using flow cytometric analysis, wherein the measurement parameters of the flow cytometer were set as FSC voltage (E00), FSC amplification gain (6), SSC voltage (350), and threshold of FSC (52); mean fluorescent index (MFI) of microbeads and nanobeads were detected; and data of nanobeads between fluorescence channel of 250 and 1023 were selected to remove background signals between fluorescence channel of 0 and 250;

FIG. 3B illustrates comparison of amounts of nanobeads in detecting PE-labeled mouse IgG₂ₐ, wherein flow cytometer parameters were set as FSC (E01), threshold of FSC (600), and SSC (350);

FIGS. 4A and 4B show immunoglobulin G (IgG) competition on anti-IgG affibody-conjugated 85-nm beads:

FIG. 4A illustrates competition of mouse IgG₂ₐ (a monoclonal antibody against VEGFA) against PE-labeled mouse IgG₂ₐ (PE-IgG) immobilized sites on anti-IgG affibody-conjugated 85-nm beads, wherein immobilized PE-IgG (10 μg/mL) was captured and consequently coupled on the beads by 30 mM EDC (pH 4.5); and the error bars represent means ±s.d;

FIG. 4B illustrates the binding intensity of PE-labeled IgG₂ₐ on anti-IgG affibody-conjugated 85-nm beads which were already immobilized with various concentrations of mouse IgG₂ₐ by 30 mM EDC (pH 4.5). The error bars show means ±s.d.;

FIGS. 5A and 5B show VEGFA detection by nanobead- and microbead-based sandwich immunoassay:

FIG. 5A illustrates mean fluorescent index of human VEGFA detected on monoclonal antibody-coated anti-IgG affibody-conjugated 85-nm beads (solid circle) and anti-VEGFA micrometer beads (open circle) measured by flow cytometer (FACScan, BD), wherein the amplification voltages for FSC, SSC and FL2 parameters of the flow cytometric analysis were set as E01, 350 and 500, respectively; the threshold of FSC was set as 600; and the mean fluorescent index of nanobeads was collected from fluorescence channel of 583 and 619; and

FIG. 5B illustrates the relative of mean fluorescent index and VEGFA concentration detected by anti-IgG affibody-conjugated 85-nm beads.

### DETAILED DESCRIPTION OF THE PREFERRED EMBODIMENTS

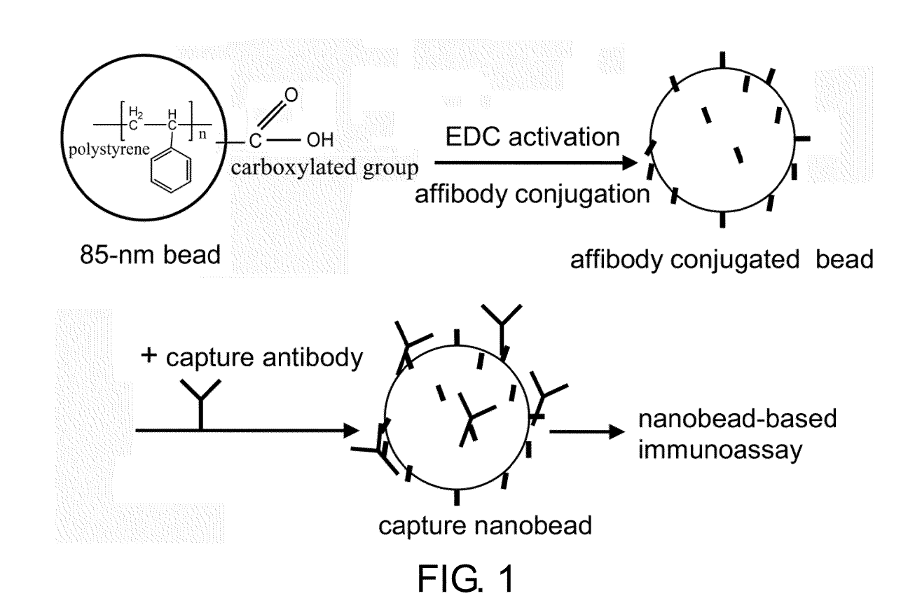

In the following examples, anti-IgG affibody-conjugated nanobeads with 85-nm carboxyl polystyrene beads were prepared and applied in a bead-based sandwich immunoassay for detecting VEGFA, which is illustrated in FIG. 1. The preparation of anti-IgG affibody-conjugated nanobeads included two steps; first step was the activation of carboxyl groups on bead surface by EDC, a carbodiimide that activates a carboxyl group to form O-acylurea and then reacts with an amine to form an amide bond, and the second step was the conjugation of anti-IgG affibody molecules on the EDC-activated bead surface. Anti-IgG affibody molecules are commercially available and known to be genetically engineered from protein A, a membrane protein of Staphylococcus aureus which binds specifically to the Fc fragment of IgG (Curr. Opin. Struct. Biol., 1995; 5: 471; Biochemistry, 1981; 20: 2361). The affibody molecule is a triple-helix polypeptide consisting of 56 amino acids with a cysteine in the N-terminal, naturally forming a dimer with 14 kDa molecular weight.

### EXAMPLE

Materials and Methods

1. Materials

Polybead® carboxylate 0.1 μm and 6 μm microspheres, used as nanobeads in the following examples, were purchased from Polysciences Inc. (Warrington, Pa.). 1-Ethyl-3-(3-dimethylaminopropyl)carbodiimide hydrochloride (EDC) was purchased from Merck. Phycoerythrin (PE) mouse IgG₂ₐ, kappa isotype control, was from eBioscience, Inc. Anti-IgG affibody® molecule was purchased from Affibody AB (Sweden). Ethylene glycol and PBS were of analytical grade. FlowCytomix human VEGF-A simplex kit and human basic kit were purchased from Bender MedSystems GmbH (Austria). Monoclonal anti-human VEGF antibody (mouse IgG₂ₐ) was purchased from R&D systems, Inc. FACScan flow cytometer (BD Biosciences) and ELISA reader (SpectraMax M2e, Molecular Devices) were used for fluorescent intensity measurement. Observation of nanobeads was done with scanning transmission electron microscope (STEM, JEM-2100F CS, JEOL, Japan) and TEM (Hitachi, HF-2000, Japan), and zeta potential was measured by Zetasizer NS90 (Malvern Instruments, Malvern, UK).

2. Conjugation of Anti-IgG Affibody on Nanobeads

Anti-IgG affibody-conjugated nanobeads were prepared by activation of Polybead® carboxylate 0.1-μm microspheres (85-nm diameter as data sheet indicated) with 75 mM EDC in ethylene glycol at 35° C.±2° C. for 24 hours (hrs) on a back-forth shaker at 80 rpm, and then the activated beads was conjugated with 100 μg/ml anti-IgG affibody molecules as described above in PBS (containing 60% ethylene glycol) at 35° C.±2° C. on a 15 rpm back-forth shaker for 2 hours and incubated at 4° C. overnight.

Briefly, 10 μL of the nanobeads was suspended in 200 μL of ethylene glycol with 75 mM EDC, whose final concentration was 0.125% solids (containing 260 μg beads) of nanobeads in a round-bottom glass tube. The EDC activation was done by back-and-forth shaking at 80 rpm (revolution per minute). After EDC activation, EDC residues were discarded by collecting nanobeads in a 1.5-ml centrifuge tube at 10,000 rcf (relative centrifugal force) for 1 hour. The EDC-activated nanobeads were stored in ethylene glycol at 4° C. Subsequently EDC-activated nanobeads (0.012% solids) were conjugated with 100 μL of 100 μg/ml anti-IgG affibody molecules solution (PBS, pH 7, containing 60% ethylene glycol) in a round-bottom glass tube for 2 hours at 35° C.±2° C. and 15 rpm back-forth shaking, then incubated at 4° C. overnight. After conjugation, affibody molecule residues were removed by spinning down nanobeads at 5000 rcf for 30 minutes and affibody-conjugated nanobeads were washed with PBS (4° C.) three times. The affibody-conjugated nanobeads were stored in PBS (4° C.) before use.

3. IgG Binding Intensity on Nanobeads and Microbeads

The binding of antibody (IgG) on 85-nm beads and 6-μm beads were via chemically coupling or capturing PE-labeled mouse IgG₂ₐ (PE-IgG) on EDC-activated beads or anti-IgG affibody-conjugated beads, respectively. For analysis of PE-IgG coupling on beads, EDC-activated beads were prepared by activation of beads (0.125% solid, containing 260 μg beads) with 75 mM EDC (in 200 μL ethylene glycol) at 35° C.±2° C. on a back-forth shaker at 80 rpm for 24 hours. These EDC-activated beads (26 μg) were conjugated subsequently with 50 μL of varied concentrations of PE-IgG (PBS, containing 60% ethylene glycol) at 4° C. overnight. For analysis of PE-IgG capturing on the affibody-conjugated beads, EDC-activated beads (26 mg) were conjugated with 100 mL of 100 mg/ml anti-IgG affibody molecules at 35° C.±2° C. and 15 rpm back-forth shaking for 2 hours and then incubated at 4° C. overnight. These anti-IgG affibody-conjugated beads were collected, washed out from conjugation solution and finally incubated with 50 μL of varied concentrations of PE-IgG at 4° C. overnight. Relative fluorescent unit (RFU) of PE-IgG on each bead sample containing 26 μg beads was suspended in a 384-well black plate and measured using a ELISA reader (SpectraMax M2e, Molecular Devices) with excitation of 488-nm wavelength light and detection of emitted light at 580-nm wavelength.

For more specific analysis of PE-IgG binding efficiency on anti-IgG affibody-conjugated beads, 50 μL of various concentrations of PE-IgG were incubated with anti-IgG affibody-conjugated 85-nm beads (2.6 ng or 26 ng) and 6-μm beads (26 ng) for 2 hours at room temperature. The PE-IgG captured beads were suspended in PBS, transferred to a plastic round-bottom tube and measured by a FACScan flow cytometer with excitation of 488-nm wavelength and detector of emitted wavelength from 564 to 606 nm.

4. Characterization of Bead Surface by Zeta Potential

Zeta potential of beads was measured to study the change of surface charge on beads in the process of EDC activation, anti-IgG affibody and capture antibody conjugation as well as capture antibody coating. The beads were suspended in 1 ml PBS, moved to a sample cell and measured using Zetasizer NS90 (Malvern Instruments, UK) at room temperature. The bead sample preparation: EDC-activated beads were prepared by incubating beads (0.125% solid, 260 μg) with 75 mM EDC in ethylene glycol at 35° C. on a back-forth shaker at 80 rpm for 24 hours. The EDC-activated beads (26 μg) were further reacted with 100 μL of 100 μg/ml anti-IgG affibody molecules at 35° C. on a back-forth shaker at 15 rpm for 4 to 6 hours and at 4° C. overnight. Monoclonal antibody-coated anti-IgG affibody-conjugated nanobeads were prepared by incubating 50 μL of 26 μg/ml monoclonal anti-human VEGFA antibody with anti-IgG affibody-conjugated nanobeads.

5. Bead Observation by Transmission Electron Microscopy

For TEM observation, 85-nm polystyrene beads and anti-IgG affibody-conjugated 85-nm beads were suspended in H₂O and 0.2 mM PBS respectively and dropped on carbon-coated 200-mesh copper grids. After air-drying at room temperature for 24 hours, the beads were observed using scanning transmission electron microscope (STEM, JEM-2100F CS, JEOL) and TEM (Hitachi, HF-2000, Japan).

6. Bead Analysis Using Flow Cytometry

Fluorescence intensity of FITC-labeled nanobeads and affibody conjugated nanobeads which were bound to PE-IgG was measured by fluorescence-assisted cell sorter (FACS), FACScan flow cytometer (BD Biosciences) equipped with a laser at 488 nm and two band pass emission filters at 515 to 545 nm and 564 to 606 nm. For each measurement a total number of 2000 to 10,000 events were recorded and flow rates for nanobeads were set at 60 μL per minute to collect data including values of side scatter channel (SSC), forward scatter channel (FSC), emitted mean fluorescence intensity (MFI) of FL-1 and FL-2. FSC and SSC amplification voltages were set as E01 and 350, respectively. FL-1 and FL-2 amplification voltages were set between 350 and 500 to trigger photomultiplier tubes (PMTs) for collecting FITC and PE fluorescence signals, respectively. All of FSC, SSC, FL-1 and FL-2 were set to logarithmic amplification and data were displayed as dot plots of FSC-SSC, and histograms of SSC, FL-1 and FL-2.

Basically, FSC value is indicative of bead volume, while SSC value is indicative of complexity and granularity of beads. Summit 5.2 software (Backman Coulter, Inc.) was used for processing data of flow cytometry.

7. VEGFA Detection by Bead-Based Sandwich Immunoassay

The anti-IgG affibody-conjugated beads were studied for detection of human vascular endothelial growth factor A (VEGFA). First, monoclonal anti-human VEGFA antibody (mouse IgG₂ₐ) was coated on anti-IgG affibody-conjugated nanobeads and each sample of 4.55×10⁷ coated nanobeads (26 ng, calculated according to datasheet information) was incubated with 50 μL of serial dilution concentrations of VEGFA (diluted with PBS containing 1 mg/mL BSA and 10 mg/ml glycine) at R.T. (25 to 28° C.) for 3 hours. The mixture solution was centrifuged at 5000 rcf for 15 minutes at 4° C., and 40 μL supernatant was discarded. Each sample was incubated with 50 μL of 25 ng/mL biotin-conjugated anti-VEGFA polyclonal antibody at room temperature for 2 hours; then 50 μL of 10 μg/ml streptavidin-labeled PE was added to label the polyclonal antibody. The bead complexes were collected using centrifuge at 5000 rcf for 15 minutes at 4° C. The bead complexes were measured by the FACScan flow cytometer as described above.

### Example 1

### Preparation of Anti-IgG Affibody-Conjugated Beads

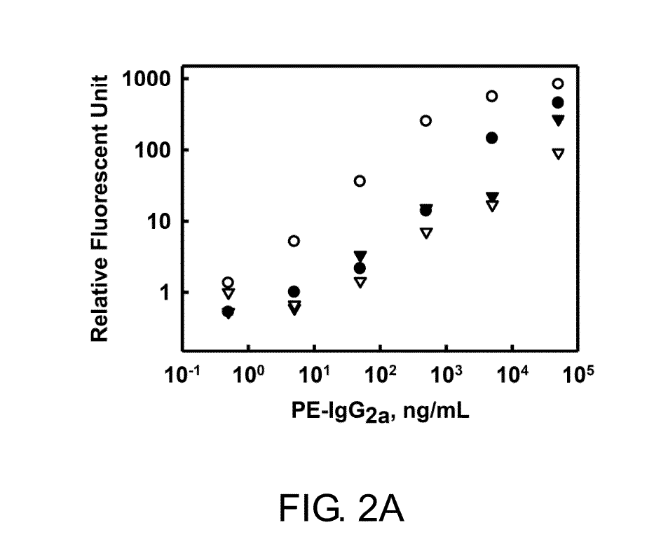

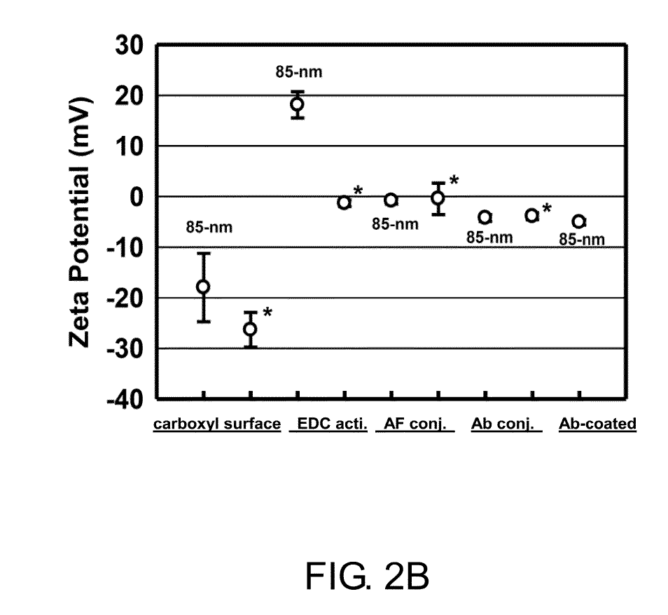

In the process of preparation of anti-IgG affibody-conjugated beads, the beads were evaluated including fluorescent IgG binding capacity on 85-nm beads compared with 6-μm beads as indicated in FIG. 2A, bead surface monitored by zeta potential in FIG. 2B. The binding capacity or intensity on bead surface was accessed with PE-labeled mouse IgG₂ₐ (PE-IgG), one species of immunoglobulin (Ig) and binding specifically to anti-IgG affibody molecule. FIG. 2A indicates that anti-IgG affibody-conjugated 85-nm beads captured PE-IgG more effectively than anti-IgG affibody-conjugated 6-μm beads, both using the same weight of beads (2.6 μg). The 2.6 μg weight of 85-nm and 6-μm beads had 1.03×10⁸ and 2.19×10⁶ μm² of bead surface respectively. The concentration of PE-IgG for saturating anti-IgG affibodies on the beads would be higher than 50 μg/ml properly, indicating the total amount of IgG molecules were settled down on the 85-nm beads with larger total surface area than that of the equal mass amount of 6-μm beads. The limitation of the 85-nm beads using an ELISA reader for IgG detection was as low as 0.5 ng/ml, in which the relative fluorescence unit was very close to background signal of empty well.

Besides, for IgG immobilization on bead surface, the chemical coupling method was not as sufficient as capturing method via anti-IgG affibody molecules. The oriented IgG molecules via anti-IgG affibody molecules were more intensive than random coupled IgG molecules on the nanobeads. These results indicate that anti-IgG affibody-conjugated 85-nm beads could generate a good yield of bead preparation and the efficacy for binding IgG molecules. During the process of bead preparation, zeta potential values of beads in PBS were measured by a Zetasizer NS90 (Malvern Instruments, Malvern, UK). After EDC activation, the zeta potential values of 85-nm beads and 6-μm beads changed dramatically from −17 mV and −26 mV to 18 mV and −1 mV, respectively, as shown in FIG. 2B. When subsequently conjugating with anti-IgG affibody molecules or antibodies on the EDC-activated bead surface, the bead surface charges shifted back to negative slightly. The change of zeta potential toward positive was due to the decrease of carboxyl groups on bead surface after EDC activation. The EDC-activated beads were not only prepared, but also stored in ethylene glycol. In ethylene glycol for a couple of weeks, the EDC activated beads still maintained the similar zeta potential value. While, EDC in acidic aqueous solution hydrolyzes easily, but acts more actively (Anal. Biochem., 2002; 305: 135).

By observing under TEM, anti-IgG affibody-conjugated 85-nm beads dispersed well in PBS (2 mM) and did not form aggregation, although the zeta potential value of these nanobeads was relatively less negative, compared to the original carboxyl functional 85-nm beads.

### Example 2

### Efficient Binding of PE-IgG on Anti-IgG Affibody-Conjugated Nanobeads

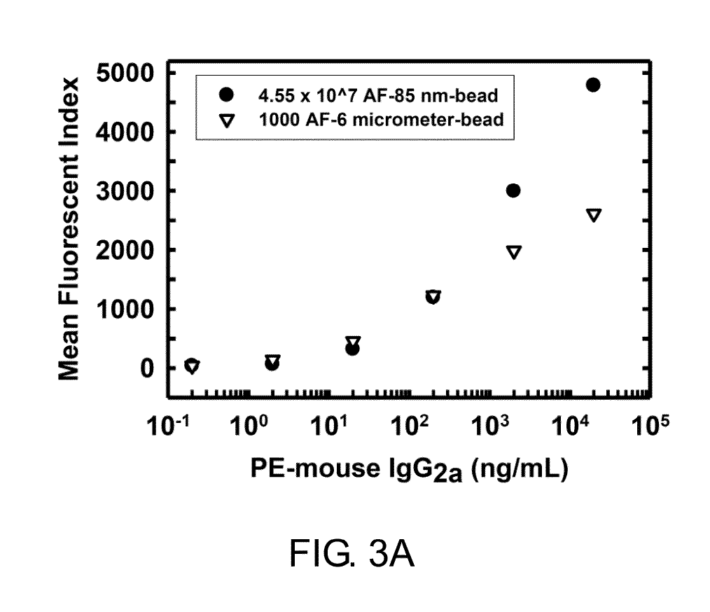

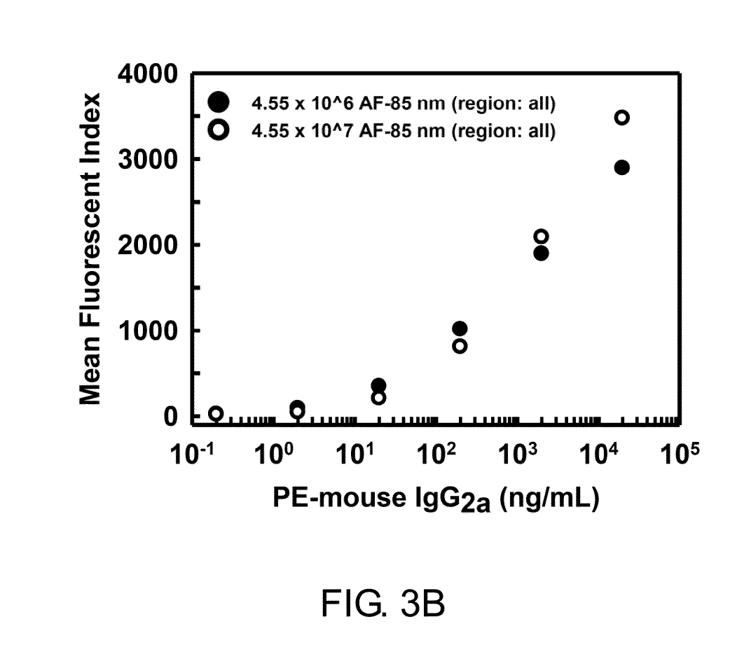

Instead of an ELISA reader, a flow cytometer, FACScan, was used for studying PE-IgG intensities on single nanobeads, as shown in FIG. 3A. PE-IgG concentrations as low as 0.2 ng/mL could still be detected by anti-IgG affibody-conjugated 85-nm beads. With the same bead surface as the 85-nm beads, anti-IgG affibody-conjugated 6-μm beads also had the same property in detection of PE-IgG Table 1 presents the calculation of surface areas of both beads.

TABLE 1
Comparison of 85-nm beads and 6-μm beads

| diameter | bead weight | bead | surface area | volume |
|---|---|---|---|---|
| (μm) | (ng) | account^{a} | (μm²) | (μm³) |
| 0.085 | 26 | 4.55 × 10⁷ | 1.03 × 10⁶ | 1.46 × 10⁴ |
| 5.766 | 130 | 1.05 × 10³ | 1.10 × 10⁶ | 1.06 × 10⁵ |

^{a}According to datasheet, 130 ng of beads is equal to 0.005 μL of 2.6% solids-latex solution

The effect of particle numbers of 85-nm beads used in PE-IgG detection is shown in FIG. 3B. Fluorescent intensities of PE-IgG molecules were slightly different but still in the similar pattern with both of 2.6 ng and 26 ng of anti-IgG affibody-conjugated 85-nm beads, which have total surface areas of 10⁵ μm² and 10⁶ μm², and bead numbers of 4.5×10⁶ and 4.5×10⁷, respectively. Less than these amounts, the 85-nm beads are very difficult to be detected by flow cytometry due to its tiny bead size.

### Example 3

### Coating of Capture Antibody on Anti-IgG Affibody-Conjugated Nanobeads

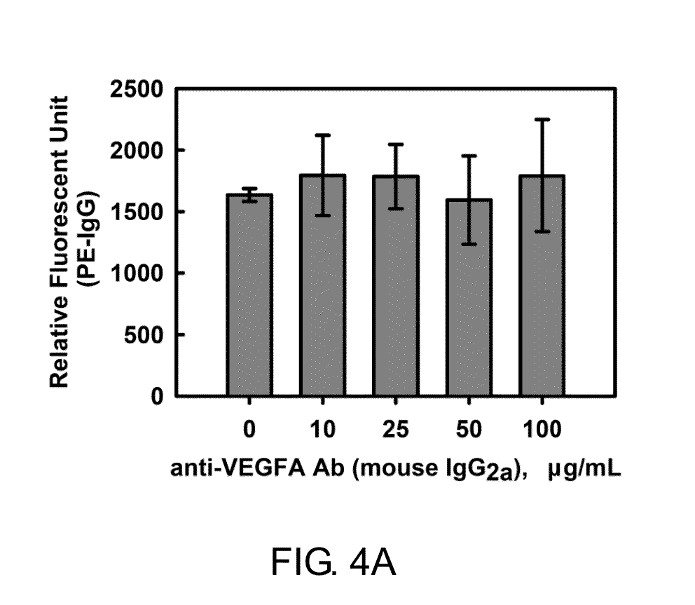

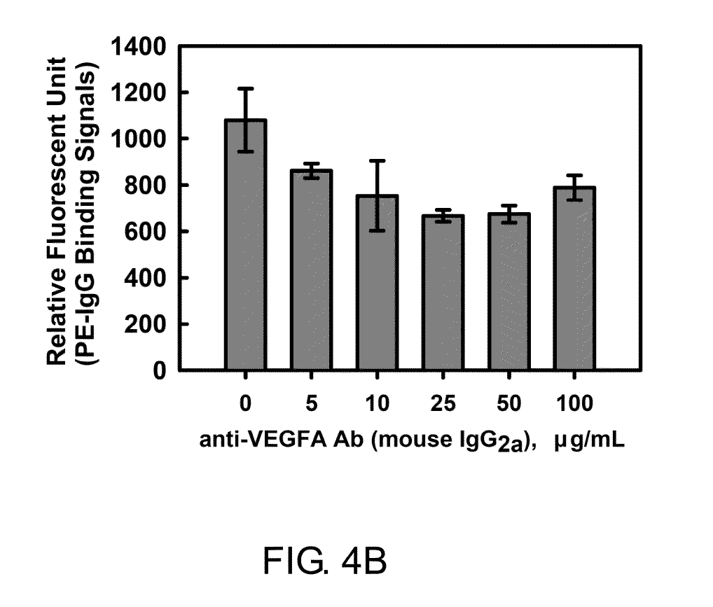

The coating concentration of capture antibody on anti-IgG affibody-conjugated 85-nm beads was evaluated by PE-IgG using a fluorescent ELISA reader. Anti-IgG affibody sites on beads were saturated by 25 μg/ml of capture antibody (mouse IgG₂ₐ) against VEGFA. This concentration (in 50 μL) provides capture antibody molecules (1.25 μg) ten times over anti-IgG affibody sites available on beads (2.6 μg of 85-nm beads, equal to 4.5×10⁹ beads). As shown in FIG. 4B, PE-IgG molecules still could bind on anti-IgG affibody-conjugated beads which were already immobilized with capture antibody. Therefore, high fluorescence intensities were detected on each capture antibody coating concentration, including 5, 10, 25, 50, and 100 μg/mL, as indicated in FIG. 4B. Capture antibody may dissociate from affibody molecules on beads immediately in EDC solution (pH 4.5) before its chemical immobilization on affibody molecules. In other words, dissociation rate of capture antibody-affibody molecule complexes might be faster than EDC coupling rate on the complexes and the sites of affibody molecules without capture antibody occupation may further bind with PE-IgG.

### Example 4

### VEGFA Detection by Nanobead-Based Sandwich Immunoassay

In a nanobead-based sandwich immunoassay, capture antibody against VEGFA, mouse produced IgG₂ₐ, was used to coat anti-IgG affibody-conjugated nanobeads since mouse IgG₂ₐ is one of the immunoglobulins with high affinity for anti-IgG affibody molecules.

For analysis of optimal coating concentration of capture antibody (mouse IgG₂ₐ) on anti-IgG affibody-conjugated nanobeads, the affibody-conjugated nanobeads were incubated with various concentrations of mouse IgG₂ₐ and subsequently detected by goat produced FITC-labeled anti-mouse antibody using flow cytometry to evaluate coating efficiency. An optimal concentration of IgG was 100 μg/ml.

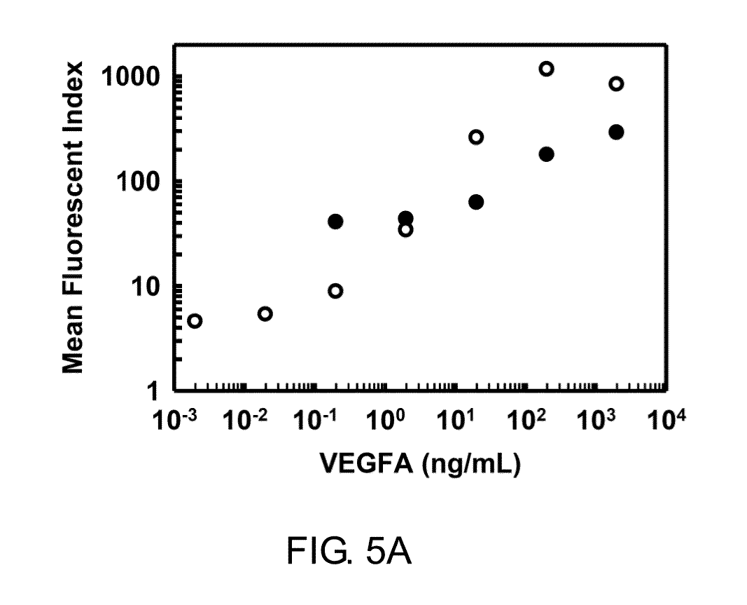

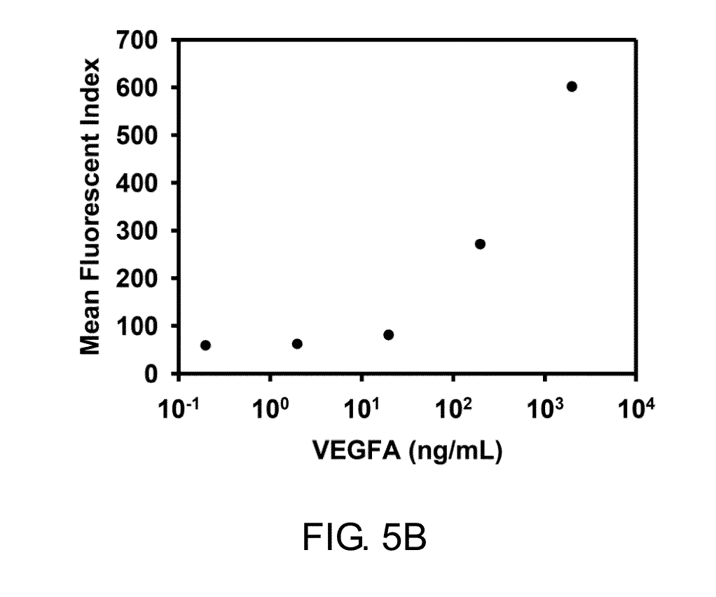

Bead-based sandwich immunoassays of VEGFA with nanobeads as well as microbeads are shown in FIG. 5A. In the nanobead-based sandwich immunoassay (FIG. 5B), the background of fluorescence intensity was significant when antigen concentration was less than detection antibody concentration. The significant background was an indicative property of anti-IgG affibody molecules, which bind not only capture antibody (IgG), but also detection antibody (IgG); both of capture antibody and detection antibody have a complete IgG structure, consisting the Fc fragment, whom protein A binds. This Fc fragment binding property is an issue for applying anti-IgG affibody molecules to immobilize capture antibody in sandwich immunoassays, although anti-IgG affibody molecules would be efficient obviously in other applications such as immunoprecipitation and antibody purification.

Thus, residues of affibody molecules without capture antibody coating may properly bind reporter antibody, labeled with fluorescent dyes; meanwhile, capture antibodies bound on anti-IgG affibody molecules may also be replaced by reporter antibodies. In previous reports, IgG surface area was close to 7 nm×5 nm measured by SEM observation, so that less than one thousand of IgG molecules is predicted to saturate on the surface of an affibody conjugated 85-nm bead. The capacity of VEGFA detection on such a tiny nanobead was between ng/ml and μg/ml.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A capture nanobead with multiple oriented adapting peptides, comprising:
a nanobead; and
multiple adapting peptides, each adapting peptide specifically recognizing a region of a capture molecule or fragment thereof and being chemically conjugated with the nanobead, whereby the adapting peptides are orientedly arranged on the nanobead.

2. The capture nanobead according to claim 1, wherein the region of the capture molecule is the constant region of IgG.

3. The capture nanobead according to claim 1, wherein the adapting peptides are anti-IgG affibodies.

4. The capture nanobead according to claim 2, which is adapted to binding for over 10 molecules of IgG per nanobead.

5. The capture nanobead according to claim 1, wherein the adapting peptides are connected to a surface of the nanobead by conjugation with a cross-linking agent.

6. The capture nanobead according to claim 2, wherein the adapting peptides are connected to a surface of the nanobead by conjugation with a cross-linking agent.

7. The capture nanobead according to claim 3, wherein the adapting peptides are connected to a surface of the nanobead by conjugation with a cross-linking agent.

8. The capture nanobead according to claim 4, wherein the adapting peptides are connected to a surface of the nanobead by conjugation with a cross-linking agent.

9. The capture nanobead according to claim 5, wherein the cross-linking agent is selected from the group consisting of 1-ethyl-3-(3-dimethylaminopropyl)carboimide hydrochloride (EDC), an epoxide, a dialdehyde, a N-hydroxysuccinimide ester, a carbodiimide, genipin, a riboflavin, a flavonoid, a 6-maleimidohexanoic acid active ester, disuccinimidyl suberate, bis(sulfosuccinimidyl)suberate, and sulfosuccinimidyl-4-(N-maleimidomethyl)cyclohexane-1-carboxylate.

10. The capture nanobead according to claim 6, wherein the cross-linking agent is selected from the group consisting of 1-ethyl-3-(3-dimethylaminopropyl)carboimide hydrochloride (EDC), an epoxide, a dialdehyde, a N-hydroxysuccinimide ester, a carbodiimide, genipin, a riboflavin, a flavonoid, a 6-maleimidohexanoic acid active ester, disuccinimidyl suberate, bis(sulfosuccinimidyl)suberate, and sulfosuccinimidyl-4-(N-maleimidomethyl)cyclohexane-1-carboxylate.

11. The capture nanobead according to claim 7, wherein the cross-linking agent is selected from the group consisting of 1-ethyl-3-(3-dimethylaminopropyl)carboimide hydrochloride (EDC), an epoxide, a dialdehyde, a N-hydroxysuccinimide ester, a carbodiimide, genipin, a riboflavin, a flavonoid, a 6-maleimidohexanoic acid active ester, disuccinimidyl suberate, bis(sulfosuccinimidyl)suberate, and sulfosuccinimidyl-4-(N-maleimidomethyl)cyclohexane-1-carboxylate.

12. The capture nanobead according to claim 5, further comprising:
capture molecules respectively binding to the adapting peptides via specifically binding of the IgG constant region.

13. The capture nanobead according to claim 12, wherein the capture molecules are chemically conjugated with the adapting peptides.

14. The capture nanobead according to claim 5, wherein the surface of the nanobead has carboxyl group; and the cross-linking agent is 1-ethyl-3-(3-dimethylaminopropyl)carboimide hydrochloride.

15. The capture nanobead according to claim 5, wherein the nanobead has a size between 10 nm to 900 nm.

16. The capture nanobead according to claim 5, wherein the nanobead has a size between 20 nm to 500 nm.

17. The capture nanobead according to claim 5, wherein the nanobead has a size between 50 nm to 200 nm.

18. The capture nanobead according to claim 14, wherein the nanobead has a size between 10 nm to 900 nm.

19. The capture nanobead according to claim 14, wherein the nanobead has a size between 20 nm to 500 nm.

20. The capture nanobead according to claim 14, wherein the nanobead has a size between 50 nm to 200 nm.
